(19) United States
(12) Patent Application Publication
(10) Pub. No.: US 2026/0201357 A1
(43) Pub. Date: Jul. 16, 2026

(54) METHOD FOR SEPARATING BETA-XYLOSIDASE ENZYME FROM AN ENZYME MIXTURE

(71) Applicant: IFP Energies Nouvelles, Rueil-Malmaison (FR)

(72) Inventors: Simon ARRAGAIN, Rueil-Malmaison Cedex (FR); Fadhel BEN CHAABANE, Rueil-Malmaison Cedex (FR)

(73) Assignee: IFP Energies Nouvelles, Rueil-Malmaison (FR)

(21) Appl. No.: 19/136,625

(22) PCT Filed: Nov. 24, 2023
(86) PCT No.: PCT/EP2023/082944

§ 371 Date: Jun. 6, 2025

(30) Foreign Application Priority Data
Dec. 8, 2022 (FR) FR2213029

(51) Int. Cl. C12N 9/24

(52) U.S. Cl. CPC C12N 9/2402; C12Y 302/01037

(57) ABSTRACT

The present invention relates to a process for separating beta-xylosidase enzymes from a mixture of enzymes comprising beta-xylosidase enzymes and other enzymes, such that the beta-xylosidase enzymes to be separated are devoid of a histidine group, and such that said beta-xylosidase enzymes are separated from the rest of the enzyme mixture by immobilized metal ion affinity chromatography IMAC.

### TECHNICAL FIELD

The present invention relates to the production of enzymes of cellulolytic and/or hemicellulolytic type, in particular within the context of the production of sugars from cellulosic or lignocellulosic materials involving an enzymatic hydrolysis of these materials. The sugars can be used/profitably exploited as they are, or continue their conversion to alcohol, in particular to ethanol, by fermentation.

### PRIOR ART

Since the 1970s, the transformation of lignocellulosic materials into ethanol, after hydrolysis of the constituent polysaccharides into fermentable sugars, has been the subject of very many studies. Mention may be made, for example, of the reference studies by the National Renewable Energy Laboratory (Process Design and Economics for Biochemical Conversion of Lignocellulosic Biomass to Ethanol, Humbird et al., NREL/TP-5100-57764, May 2011).

Lignocellulosic materials are cellulosic materials, i.e. materials consisting of cellulose, hemicellulose, which are polysaccharides essentially consisting of pentoses and hexoses, and also lignin, which is a macromolecule of complex structure and of high molecular weight, based on phenolic compounds. For the sake of brevity, they will be grouped together in the present text under the generic term “biomass”.

Wood, straw and corn cobs are the lignocellulosic materials most commonly used, but other resources, dedicated forestry crops, residues from alcohol-yielding, sugar-yielding and cereal plants, products and residues from the paper industry and products from the transformation of lignocellulosic materials are usable. They are for the majority constituted of about 35% to 50% of cellulose, 20% to 30% of hemicellulose and 15% to 25% of lignin.

The process for the biochemical transformation of the lignocellulosic materials to sugars, and then optionally to alcohol of ethanol type, comprises a physicochemical pretreatment step, followed by a step of enzymatic hydrolysis using an enzyme cocktail. It may be followed by a step of ethanolic fermentation of the sugars released, the ethanolic fermentation and the enzymatic hydrolysis possibly being conducted simultaneously, and then by a step of purification of the ethanol. One example of such a process converting biomass to ethanol is described in patent EP 3 484 945, to which reference can be made for further details.

The enzyme cocktail used for the hydrolysis is a mixture of cellulolytic enzymes (also known as cellulases) and/or hemicellulolytic enzymes. Cellulolytic enzymes have three major types of activities: endoglucanases, exoglucanases and cellobiases, the latter also being known as β-glucosidases. Hemicellulolytic enzymes in particular have xylanase activities.

The most used cellulolytic microorganism for the industrial production of the enzyme cocktail is the fungus Trichoderma reesei. The wild-type strains have the faculty of secreting, in the presence of a carbon-based inducer substrate, for example cellulose, the enzyme cocktail considered as being the best suited for the hydrolysis of cellulose. Other proteins possessing properties vital for the hydrolysis of lignocellulosic materials are also produced by Trichoderma reesei, xylanases for example. The presence of a carbon-based inducer substrate is essential for the expression of the cellulolytic and/or hemicellulolytic enzymes. The nature of the carbon-based substrate has a strong influence on the composition of the enzyme cocktail. This is the case for xylose, which, when combined with a carbon-based inducer substrate such as cellulose or lactose, makes it possible to significantly improve the “xylanase” activity.

More specifically, within the context of the production of “second generation” (2G) bioethanol from lignocellulosic biomass, one of the main challenges is that of degrading the cellulose and hemicellulose fibers by virtue of a pretreatment of the biomass (treatment, for example, with an acidic or basic liquor, and then cooking or steam explosion) and then by the action of cellulolytic and hemicellulolytic enzymes which depolymerize the fibers. Cellobiohydrolases (CBH1 and CBH2) make it possible to produce oligomers of sugar such as cellobiose, cellotriose and other oligomers of glucose produced from cellulose. β-Glucosidase makes it possible to degrade cellobiose (and also the other oligomers) into glucose which is directly assimilable by a yeast for the production of bioethanol. The degradation of hemicelluloses is carried out by xylanases or xylobiohydrolases, and enables the formation of oligomers of xylose (xylobiose, xylotriose, and other oligomers of xylose). The action of β-xylosidase makes it possible to degrade these oligomers of xylose to produce xylose. Xylanases are generally inhibited by xylobiose and short xylooligosaccharides, and the lack of β-xylosidases is then responsible for the rate-limiting step in the hydrolysis of xylan.

Patent application WO 2011/079048 teaches that, in a process for the simultaneous hydrolysis and fermentation of biomass (SSF, for Simultaneous Saccharification and Fermentation), increasing the beta-xylosidase activity of the enzyme cocktail used for the enzymatic hydrolysis has a beneficial effect on the enzymatic hydrolysis of certain biomasses since it makes it possible to reduce the amount of enzymes needed. It also makes it possible to hydrolyze alkyl xylosides.

It is therefore advantageous to isolate the beta-xylosidases present in enzyme mixtures produced by microorganisms, for example in order to enrich a given enzyme cocktail with beta-xylosidases. To do this, various techniques have already been proposed, in particular for first of all separating, in the culture medium, the fungus from the enzymes that it has produced. Thus, patent U.S. Pat. No. 3,398,055 teaches the separation and purification of cellulases produced by the fungus Trichoderma reesei: The fungus is separated from the enzymes by filtration with a rotary filter under vacuum. The enzymes are then separated by passing them through a column using cotton, and by eluting them with a basic solution.

Patent WO 2018/015228 proposes separating the enzymes from the fungus via a succession of steps of treatment of a culture medium, including a step of filtration of the culture medium through a filter press, and then a step of tangential microfiltration of the liquid phase obtained.

It is also known to separate beta-xylosidase from an enzyme mixture by fractional precipitation with ethanol, as described in the publication by V. Cortez et al. “Xylanase and β-xylosidase separation by fractional precipitation”, Process Biochemistry, Volume 35, Issues 3-4, 1999, Pages 277-283. This is an advantageous technique, but it is not devoid of drawbacks, insofar as it requires the use of a solvent and necessitates numerous steps, making it costly and complex to implement.

An object of the invention is then to develop an improved technique for separating enzymes from a mixture of enzymes, and more particularly a technique for separating beta-xylosidases in a mixture containing beta-xylosidases and other types of enzymes. It is more particularly directed to a separation technique which is highly efficient and deployable on an industrial scale.

### SUMMARY OF THE INVENTION

A first subject of the invention is a process for separating beta-xylosidase enzymes from a mixture of enzymes comprising beta-xylosidase enzymes and other enzymes, such that:
the beta-xylosidase enzymes to be separated are devoid of a histidine group,
and such that said beta-xylosidase enzymes are separated from the rest of the enzyme mixture by immobilized metal ion affinity chromatography (hereinafter also denoted by its acronym IMAC, for Immobilized Metal Affinity Chromatography).

IMAC chromatography is known for separating proteins possessing histidine groups exposed on their surface, whether naturally or as a result of genetic modification; in the latter case reference is made to a histidine “tag” or histidine “cluster” added to the protein. Reference may also be made to the publication by V. Gaberc-Porekar et al. “Perspectives of immobilized-metal affinity chromatography”, J Biochem. Biophys. Methods. 2001 October 30; 49(1-3) 335-60.

Now, utterly surprisingly, it has been found, within the context of the present invention, that this chromatography technique was nevertheless capable of separating enzymes devoid of a histidine group, and very particularly the beta-xylosidases that the inventors sought to separate in an enzyme cocktail produced by microorganisms.

This is very advantageous in a number of respects:
beta-xylosidase enzymes can be isolated by this technique without having to modify them beforehand to have these histidine tags or clusters. By avoiding modifying them, the manner of obtaining/separating them is of course simplified by eliminating a genetic modification step. But any risk of performance loss due to a modification of their behavior/alteration of their activity due to the presence of these histidine groups is also limited (numerous cases have been described in the literature of enzymes that have had their activity altered following the addition of a histidine tag, especially in the case of metalloenzymes and multimeric enzymes),
the technique of separation by IMAC chromatography is highly efficient: it can be deployed on an industrial scale, the materials needed to perform this type of chromatography are stable and can therefore be stored without the risk of degradation, the elution conditions are generally not severe, the reactants employed are generally reusable, which makes it economically advantageous, and the results thereof in terms of selectivity in the enzymes separated, in particular in this case the beta-xylosidases, are excellent,
the separation can be carried out in a single step, resulting in a process that is easier and more rapid to implement.

Generally, the other enzymes of said mixture may comprise at least one enzyme chosen from cellulases, hemicellulases, and/or from hemicellulases.

Generally, the other enzymes of said mixture may comprise beta-glucosidases, endoglucanases, and possibly cellobiohydrolases.

The beta-xylosidases may constitute at least 1% by weight, in particular between 2% and 15% by weight or between 3% and 8% by weight, of all of the enzymes present in the mixture. This the content generally encountered in the enzyme cocktails produced by Trichoderma reesei, but, of course, the invention applies in the same way to enzyme mixtures containing a greater proportion of beta-xylosidases.

Preferably, the immobilized metal ion affinity chromatography IMAC uses:
a solid immobile phase comprising a matrix on which metal ions are fixed by chelating agents,
and a liquid mobile phase referred to as eluent.

The matrix of the immobile phase may advantageously be chosen from at least one of the following compounds: agarose gel, crosslinked dextran gel, silica.

The chelating agents may advantageously be chosen from at least one of the following compounds: iminodiacetic acid IDA, nitrolotriacetic acid NTA, tris[carboxymethyl]ethylenediamine TED.

The metal ions may advantageously be chosen from: metal ions of transition metals, in particular chosen from
the divalent ions Cu(II), Ni(II), Zn(II), Co(II),
trivalent metal ions, in particular chosen from the trivalent ions Fe(III), Al(III), Ga(III),
or tetravalent metal ions, in particular the metal ion Zr(IV).

According to a preferred embodiment of the invention, the enzyme mixture is obtained from the production of enzymes by a microorganism, in particular by a filamentous fungus, for example of the genus Trichoderma, in particular the species Trichoderma reesei.

The separation process according to the invention may comprise a preliminary step of separating a culture medium comprising the enzyme mixture and a microorganism that produced said mixture, said preliminary step being aimed at separating the microorganism from said enzyme mixture and comprising in particular a filtration or several successive filtrations of the culture medium. This preliminary separation may for example be carried out as described in the patent WO 2018/015228. Thus obtained, after solid/liquid separation, are firstly the microorganism that produced the enzymes (which is also referred to as must) in solid/semi-solid form and, secondly, the soluble enzymes in liquid (aqueous) phase. This liquid phase may optionally be concentrated, and then it will be able to be treated according to the invention.

The separation process according to the invention may also comprise a step of treating the must, which may or may not have been separated from the rest of the culture medium, said treatment comprising cooling the must and then separating the must and an “additional” liquid phase containing an additional amount of enzyme mixture, as taught in the patent EP 3 174 979.

If this additional separation is performed on the must that has already been separated, the liquid phase obtained after the solid/liquid separation described above can then be mixed with this additional liquid phase, and the process according to the invention can be performed on the mixture of these two liquid phases.

The process according to the invention may naturally be performed on a liquid phase containing the enzyme mixture that has been concentrated beforehand.

According to a first variant of the separation process of the invention, the chromatography is carried out continuously in a chromatography column containing a solid immobile phase through which a liquid mobile phase referred to as eluent can be continuously passed.

According to another variant, the separation by chromatography according to the invention is carried out batchwise, by bringing an immobile chromatography phase into contact with the mixture comprising beta-xylosidase enzymes and other enzymes, in a liquid medium, to constitute a reaction medium in a container for a given duration, and then by eluting the solid part of said reaction medium in order to extract the beta-xylosidases therefrom.

In this variant, the separation may comprise a step of mixing the immobile phase with the mixture of enzymes in solution, then an optional decantation step, then a step of isolating the solid phase from the reaction medium, then an optional washing step, then a step of eluting the isolated solid phase in order to extract the beta-xylosidases therefrom.

The separation by chromatography according to the invention fixes the beta-xylosidases on the immobile phase preferably at a pH of between 6.5 and 9, and the beta-xylosidases are preferably eluted by changing the nature, the composition or the concentration of the eluent, which makes it possible, in particular, to change the pH of the immobile phase.

A subject of the invention is also the beta-xylosidase enzyme, produced in particular by a fungus such as Aspergillus or Trichoderma, and in particular obtained by the separation process as described above, and which has a specific activity of at least 10 μmol of p-nitrophenol·min⁻¹·mg⁻¹ of enzyme, in particular of at least 20 or at least 30 or at least 35 μmol of p-nitrophenol·min⁻¹·mg⁻¹ of enzyme. This is a high specific activity, which demonstrates efficient separation resulting from a high purity of the beta-xylosidase enzyme thus separated.

The method for measuring specific activity, known to those skilled in the art, consists in placing the purified enzyme in the presence of PNP-Xylose (p-nitrophenyl-β-D-xylopyranoside). Under the action of beta-xylosidase, the PNP released is monitored by spectroscopy and the specific activity is calculated using a PNP standard range.

One example of beta-xylosidase targeted by the present invention is a xylan 1,4-beta-xylosidase protein obtained from Trichoderma reesei with the reference XP_006964075.1 in NCBI (acronym for National Center for Biotechnology Information) and described in the Uniprot database under the reference Q92458_HYPJE (EC: 3.2.1.37; taxonomic identifier 51453 NCBI; sequence version 2 of Jan. 6, 1998, Gene: bxl1, -organism: Hypocrea jecorina (Trichoderma reesei)).

Also targeted are all of the beta-xylosidases with sequences having at least 50% identity with this beta-xylosidase, in particular at least 60% or at least 65% or at least 80% or at least 85% or at least 90% or at least 95% or at least 98 or 99% with this beta-xylosidase.

The invention more generally targets any beta-xylosidase that may be obtained in particular with a fungus of the genus Trichoderma, in particular the species Trichoderma reesei or citrinoviride or orientale or longibrachiatum or arundinaceum, or with a fungus of the genus Aspergillus, in particular the species Aspergillus niger, japonicus, oryzae, clavatus, aculeatus, awamori, flavus.

A subject of the invention is also the beta-xylosidase enzyme, in particular obtained by the separation process described above, which has a purity of greater than or equal to 90%, generally greater than or equal to 95% or 97%.

The purity was evaluated, in a known way, by electrophoresis on SDS-PAGE gel (a polyacrylamide gel containing sodium dodecyl sulfate), and then analyzed with the Image-Lab software, available from Bio-Rad.

An enzyme is thus obtained which is highly pure, which makes it highly profitably exploitable. This result is all the more remarkable since the separation according to the invention can be performed on enzyme cocktails possibly containing tens or even around a hundred different enzymes, such as those produced by microorganisms such as Trichoderma, for example.

A subject of the invention is also the use of the beta-xylosidase enzymes, in particular obtained according to the process described above, for enriching an enzyme mixture produced by a microorganism with beta-xylosidase enzymes.

It is thus possible to add them, in controlled fashion, to a process for converting different types of lignocellulosic biomass to sugar(s) (saccharification including enzymatic hydrolysis of the biomass) or into alcohol (saccharification and fermentation) having various recalcitrances to sugars or to alcohol (ethanol type).

Another use consists in profitably exploiting these beta-xylosidase enzymes as they are, for applications specifically requiring beta-xylosidase activity.

The beta-xylosidase may be purified and sold in pure form for biotechnological applications, whether for degrading or for producing xylooligosaccharides.

The beta-xylosidase may be added to a beta-xylosidase-poor enzyme cocktail for industrial applications in the field of the degradation of lignocellulosic biomass for the purpose of producing sugars that can be profitably made use of in bioproducts or for the production of alcohols including in particular bioethanol.

### LIST OF THE FIGURES

FIG. 1 represents the FPLC profile (acronym for Fast Protein Liquid Chromatography, which is a known technique for rapid chromatography of proteins in liquid phase) for the separation of the beta-xylosidase from an enzyme mixture according to one exemplary embodiment of the invention.

FIG. 2 represents the results of electrophoresis with an SDS-PAGE gel of the beta-xylosidase after FPLC purification (described below).

FIG. 3 represents a graph of the activities of the beta-xylosidases separated according to 2 exemplary embodiments of the invention, in the form of bar charts, with the identification of the examples 1 and 2 on the x-axis and their activities expressed in μmol of p-nitrophenol·min⁻¹·mg⁻¹ of enzyme on the y-axis.

### DESCRIPTION OF THE EMBODIMENTS

The invention will be described hereinafter in detail, with the aid of the figures and examples which are given by way of illustration and are therefore in no way limiting.

The invention proposes a process making it possible to separate a particular enzyme from a mixture of enzymes: the beta-xylosidase enzyme.

The invention is more particularly directed to separating this enzyme from an enzyme cocktail produced by a microorganism, more particularly by the fungus Trichoderma, in particular Trichoderma reesei, to which the following examples and detailed descriptions relate.

But the invention applies analogously to the separation of this enzyme from any mixture of enzymes containing it, and in particular any enzyme cocktail produced by microorganisms that contain this enzyme in varying proportions.

The process according to the invention makes it possible to simply and rapidly purify β-xylosidase from T. reesei irrespective of the type of T. reesei strain used.

The process is a process for purifying an enzyme of interest (beta-xylosidase) from a complex mixture of enzymes (around a hundred enzymes) carried out in a single step. This requires producing the enzymes and separating the mycelium beforehand.

The following description details the variant of the invention using a chromatography column operating continuously. However, the invention may also be implemented without a column, in analogous fashion, batchwise.

### Prior Steps

In order to implement the separation process according to the invention, preliminary separation of the culture medium comprising the enzyme cocktail and the fungus Trichoderma reesei is first of all carried out. To do this, the culture medium is subjected, within a period of less than 24 hours from halting the production, to a separation on a filter press lined with a fabric having a porosity of 3-20 μm, so as to obtain a filtrate having a corrected optical density OD 600 nm of less than 2.5. The liquid phase obtained is subjected to tangential microfiltration on a ceramic membrane having a cutoff threshold of between 0.5 and 1.4 μm, so that the corrected optical density OD 600 nm does not exceed 0.1. The separation on a filter press and the microfiltration are carried out at 20-30° C., preferably 22-27° C.

On conclusion of the separation on a filter press, 5-10% by weight of solid residue (“cake”) and 90-95% by weight of filtrate are generally obtained. Advantageously, the microfiltration of the filtrate obtained at the end of the filter press is carried out within a period of at most 30 hours, and preferably at most 24 hours.

Preferably, the tangential microfiltration is carried out on a ceramic membrane having a cutoff threshold of between 0.8 and 1.4 μm.

The liquid phase obtained after microfiltration may be subjected to ultrafiltration, preferably on a ceramic membrane, and even more preferably on a ceramic membrane having a cutoff threshold of between 5 and 15 kDa.

The filtration method described here adopts the teaching of the patent WO 2018/015228, to which reference will be made for further details.

The retentate obtained is then passed through an IMAC affinity column making it possible to separate the enzymes having a polyhistidine-tag (also referred to as “His-tag”). This is an amino acid motif in a protein that consists of at least six histidine residues, often inserted at the N- or C-terminus of the protein. It is sometimes denoted by the names “hexa histidine-tag” or “6xHis-tag”.

It will be clearly noted, however, that the beta-xylosidase purified from the cocktail does not comprise a histidine tag (nor do any of the other enzymes in the enzyme mixture here).

### Purification on IMAC Column

The supernatant containing the enzymes (permeate from the microfiltration or retentate from the ultrafiltration), that is to say the cellulases produced by Trichoderma reesei, is stored between 4° C. and 30° C., but preferably below 10° C.

The supernatant obtained is loaded into an immobilized metal ion affinity chromatography column, also referred to as IMAC (acronym for Immobilized Metal Affinity Chromatography). This type of affinity chromatography is based on the mechanism of chelation of immobilized metal cations. This generally makes it possible to purify proteins possessing a histidine tag from the supernatant containing a complex mixture of various proteins of biological origin.

The chelation of the (generally divalent) metal ion is a process that enables the formation of a complex between a metal cation and a ligand fixed on a solid phase. By virtue of this chelation, the metal ions remain immobilized in a column into which the enzyme mixture it is desired to fractionate or purify is poured. The bonds between the metal ion and the ligand generally form within a pH range of between 7 and 8. In order to maintain this pH, the column is equilibrated beforehand using a buffer solution.

The solution in which the sample can be solvated ideally has a high ionic strength in order to reduce the nonspecific electrostatic interactions, but these ions do not themselves need to bond with the metals. The solution is also preferably neutral or slightly alkaline, since the interactions between the histidine groups and the metals are deactivated in the presence of protons that occupy the binding sites on the amino acid. Examples of this kind of solution are 50 mM Tris-acetate (tris(hydroxymethyl)aminomethane acetate (CH3COO⁻)) or 20 to 50 mM sodium phosphate. Tris-HCl (tris(hydroxymethyl)aminomethane HCl) makes it possible to purify enzymes having quite strong interactions between the protein and the metals.

For the eluent, it is possible to choose an acidic solution with a pH gradient of from 7 to 4, in order to protonate the amino acids interacting with the IMAC matrix, which induces a drastic reduction in the affinity of the enzyme for the resin. Alternatively, it is also possible to use an imidazole solution to replace the proteins on the binding sites (to exchange the ligands). Lastly, it is also possible to extract the metal ion with a powerful chelating agent such as ethylenediaminetetraacetic acid EDTA (often used to regenerate the column).

### Production Step

The enzyme cocktail on which the separation process according to the invention is performed is produced by Trichoderma reesei in a conventional production chain, by aerated fermentation. Examples of a process for producing an enzyme cocktail via this fungus are described in the patents FR 3 024 463, FR 3 049 957, FR 3 085 961 and FR 3 088 934. An improvement in the process to increase the content of beta-glucosidase and/or beta-xylosidase by cooling the must obtained at the end of the production is described in the patent EP 3 174 979.

The process for producing the enzyme cocktail begins with a propagation phase, generally carried out in small reactors of increasing size, for the purpose of multiplying the filamentous fungus and of limiting the duration of the lag phase and the contamination risks.

When this production is judged to be sufficient (fungus concentration greater than or equal to 10 g/l, preferably greater than or equal to 15 g/l), the culture medium is transferred into the final reactor of large volume.

The enzyme production process comprises two phases, thus detailed according to a preferred embodiment:
a phase a) of growth of said microorganism in the presence of at least one carbon-based growth substrate in an aerated closed reactor, said growth phase being carried out with a carbon-based growth substrate concentration of between 10 and 90 g/l,
a phase b) of production of the enzyme cocktail, in which at least one carbon-based inducer substrate is introduced, said carbon-based inducer substrate being chosen from the group formed by lactose, cellobiose, sophorose, the residues obtained after ethanolic fermentation of the monomeric sugars of the cellulosic biomass enzymatic hydrolyzates, and/or a crude extract of water-soluble pentoses originating from the pretreatment of a cellulosic biomass, said production phase being carried out with a carbon-based production substrate concentration of between 150 and 400 g/l.

The microorganisms used in the process for producing an enzyme cocktail according to the invention are fungal strains belonging to the species Trichoderma reesei.

The most effective industrial strains are the strains belonging to the species Trichoderma reesei, which are modified to improve the enzyme cocktail by means of mutation-selection processes.

The strains improved by genetic recombination techniques may also be used. These strains are cultured in stirred and aerated reactors under conditions compatible with their growth and the production of the enzymes.

As examples of strains and methods for obtaining them, it can be recalled that the conventional genetic mutation techniques have enabled the selection of strains of Trichoderma reesei which hyperproduce cellulases, such as the strains MCG77 (Gallo—patent US 4 275 167), MCG 80 (Allen, A. L. and Andreotti, R. E., Biotechnol.-Bioeng. 1982, 12, 451-459, 1982), RUT C30 (Montenecourt, B. S. and Eveleigh, D. E., Appl. Environ. Microbiol. 1977, 34, 777-782) and CL847 (Durand et al., 1984, Proc. Colloque SFM “Génétique des microorganismes industriels” [Genetics of industrial microorganisms]. Paris. H. Heslot Ed., pages 39-50). The improvements have made it possible to obtain hyperproductive strains that are less sensitive to catabolic repression on monomeric sugars in particular, for example glucose, compared to wild strains.

Recombinant strains have also been obtained from strains of Trichoderma reesei such as Qm9414, RutC30, CL847, by cloning heterologous genes, for example the invertase from Aspergillus niger, enabling Trichoderma reesei to use sucrose as a carbon source. These strains have retained their hyperproductivity and their ability to be cultured in a fermenter.

Said carbon-based growth substrate for said microorganism that is used in said growth phase a) of the process according to the invention is advantageously chosen from industrial soluble sugars, and preferably from glucose, lactose, xylose, liquid residues obtained after ethanolic fermentation of the monomeric sugars of the enzymatic hydrolyzates of lignocellulosic materials, and extracts of the hemicellulosic fraction in the form of monomers originating from pretreated lignocellulosic substrate, used alone or as a mixture.

Depending on its nature, said carbon-based growth substrate is introduced into the closed reactor before sterilization or is sterilized separately and introduced into the closed reactor after sterilization of the latter.

Said carbon-based growth substrate is used in said growth phase a) at an initial concentration usually of between 20 and 90 g of carbon-based substrate per liter of reaction volume.

Preferably, said growth phase a) is carried out over a period of between 30 and 70 h, preferably between 30 and 40 h.

Preferably, said growth phase a) is carried out at a pH of 4.8 and at a temperature of 20-30° C., generally 22-27° C., preferably about 27° C.

Said carbon-based inducer substrate used in said production phase b) is advantageously fed in fed-batch phase mode with a limiting flow of between 30 and 80 mg per gram of cells and per hour. The temperature is generally the same as in step a).

At the end of the enzyme production step, a medium containing a solids concentration of between 10 and 45 g/l (corresponding to the dry fungus) is generally obtained; the enzymes are all water soluble. The pellet measured after centrifugation (4000 rpm, 5 minutes) is greater than 15% and often about 30%, and even up to 60%. It corresponds to the percentage of the volume occupied by the solid relative to the total volume of the sample.

The aim of the invention is to separate the fungal enzymes, then to purify the beta-xylosidase, in order to sell it or to add it specifically to a mixture of enzymes, in a biochemical process involving enzymes, if they are limiting.

The invention may also apply to any enzyme mixture produced by a microorganism, referred to as enzyme cocktail, including the enzyme cocktails resulting from a reaction medium containing the microorganisms which has been treated, in particular by cooling, as described in the patent EP3174979.

### Solid/Liquid Separation Steps in Which the Fungus is Separated From the Liquid

The liquid contains the enzymes and the residual salts.
Once the enzymes have been separated from the mycelium, the pH of the supernatant (containing the enzymes) should be adjusted within a pH range of from 6.5-9, preferentially to a pH of 8 (change of buffer via a desalting column, via filtration, via ultrafiltration, under pressure (stirred cell commercially available under the name Amicon from Merck, or ultrafiltration cell commercially available under the name Pellicon with Ultracel 10kD membrane also from Merck)
Once the cocktail of cellulases has been buffered, an IMAC column described above is used.

It should be noted that the enzyme mixtures tested have a type composition (the contents indicated are expressed abundantly and are approximate data which do however give an idea of the distribution of the enzymes in the mixture):
content of CEL7A=CBH1: approximately 35%,
content of CEL6A=CBH2: approximately 30%,
content of BGL 1=beta-glucosidase 1: approximately 5%,
content of endoglucanase I=Cel7B: approximately 10%,
content of endoglucanase II=Cel5A: approximately 10%,
content of others (enzymes and/or other compounds): approximately 10%.

For examples of analyses of secretomes of modified Trichoderma reesei strains, RUT-C30 and CL847, reference may be made to the publication “Comparative secretome analyses of two Trichoderma reesei RUT-C30 and CL847 hypersecretory strains”, from I. Herpoël-Gimbert et al., Biotechnology for biofuels, article number 18(2008) published 23 Dec. 2008, and in particular to table I therein.

The HisTrap Crude column (Cytiva, 5 mL), preloaded with the nickel ion Ni²⁺, is equilibrated to a pH of between 6.5 and 9, preferentially to a pH of 8. This type of column is available from Cytiva under the full name “His Trap FF crude histidine-tagged protein purification column”.

The equilibration buffer may be Tris, Bis-Tris, Phosphate, 4-(2-hydroxyethyl)-1-piperazineethanesulfonic acid, also referred to as HEPES, or any other buffer solution falling within the pH range between 6.5 and 9. The buffer solution may contain salts (NaCl, KCl) at between 0 -500 mM, but preferably at 50 mM.
Once the column has been equilibrated, the clarified supernatant may be filtered and then loaded into the column via a peristaltic pump, via a “Fast Protein Liquid Chromatography” system, a system which generally comprises a pump, a UV detector, a conductivity measuring device, a fraction collector and valves enabling passage in particular from one column to another. Such a system is commercially available in particular from Bio-Rad. Another chromatographic system is also available from Cytiva under the name “AKTA pure protein purification system”.

Gravity separation techniques may also be used.

The column is washed in the presence of from 0 to 40 mM imidazole, generally with 20 mM imidazole.

Surprisingly, and as has never been observed before, the beta-xylosidase from T. reesei is fixed to the solid IMAC phase and can be easily purified on this type of resin, even though it does not have any histidine tag.

In order to elute the beta-xylosidase from T. reesei, it is necessary to perform a gradient or elution in the presence of 500 mM imidazole, or to reduce the pH in order to decrease the affinity of the enzyme to the solid phase.

The beta-xylosidase enzyme from T. reesei purified under these conditions is of high purity (verification was performed by mass spectrometry), and it is active (the activity test shows that the enzyme is active on 4-nitrophenyl β-D-xylopyranoside (4-NPX) and releases para-nitrophenol pNP).

The specific activity of the beta-xylosidase is evaluated with 4-nitrophenyl β-D-xylopyranoside (4-NPX) as substrate with activities at 50° C. of between 10 and 100 μmol p-nitrophenol·min¹·mg enzymes⁻¹ and generally of at least 20 and approximately 35 μmol p-nitrophenol·min⁻¹·mg enzymes⁻¹ (or more). The same protocol is used as for measuring a beta-glucosidase activity. Only the substrate (para-nitrophenyl β-D-xylopyranoside (pNP)) is changed and beta-xylosidase is used. The principle for measuring the specific activity with this type of reagent is well known in the literature.

### EXAMPLES

### Example 1

The experiments were carried out in the laboratory, on enzyme cocktails produced by Trichoderma reesei according to the procedure described above, using the strain CL847, already cited and also described in the publication by Jourdier E. et al., “A new stoichiometric miniaturization strategy for screening of industrial microbial strains: application to cellulase hyper-producing Trichoderma reesei strains” (Microb. Cell Fact. 2012 May 30; 11:70. doi: 10.1186/1475-2859-11-70. PMID: 22646695; PMCID: PMC3434075.).

### Example 2

The experiments were carried out in the laboratory, on enzyme cocktails produced by Trichoderma reesei according to the procedure described above, using the strain described in table 1 of the patent EP 3 174 979 (SEQ ID no: 7 as nucleic acid, SEQ ID no: 8 as polypeptide) under the reference 130G9.

The description that follows relates to the treatment of the culture medium obtained in each of the two examples: The extracellular medium was separated from the mycelium by filtration. The extracellular medium containing the enzymes secreted by Trichoderma reesei was removed by filtration through a Pellicon membrane (10 kDa) and then diluted three times in buffer A Tris-Cl 50 mM pH 8, NaCl 50 mM.

This step has the double advantage of removing low-molecular-weight molecules present in the culture medium and of bringing the pH to 8. The protein extract is then centrifuged for 10 min at 5000 rpm and filtered at 0.2 μm (PES filter, VWR, 514-2073) with a syringe.

The protein solution is then loaded into the HisTrap column (Cytiva, HisTrap™ FF and His Trap Crude, 5 mL) identified above, which has an immobile phase based on crosslinked sepharose coupled to nickel ions via chelating groups.

The column is pre-equilibrated with 7 column volumes in buffer A Tris-Cl 50 mM pH 8, NaCl 50 mM. A linear gradient is then applied for 10 column volumes towards the solution of buffer B Tris-Cl 50 mM pH 8, NaCl 50 mM, imidazole 500 mM. The protein is eluted in a homogeneous and symmetrical peak. It is then concentrated and washed three times in buffer A by centrifugation at 5000 rpm with ultrafiltration units commercially available under the name Vivaspin (10 kDa) from Sartorius in order to remove the imidazole. The pure protein was then analyzed on SDS-PAGE gel and by mass spectrometry. These analyses made it possible to unambiguously demonstrate that the protein purified/separated from the rest of the enzymes of the starting cocktail is beta-xylosidase. It should be noted that, on the other hand, the CBH2s were not separated, which, however, with a histidine tag, is doubly surprising.

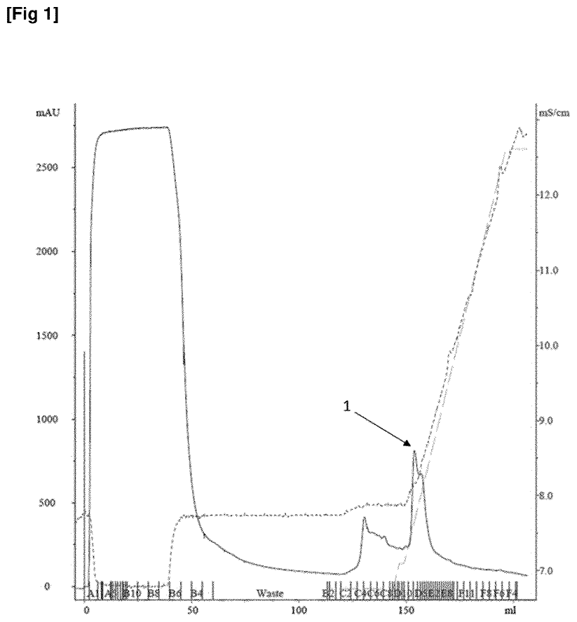

FIG. 1 represents the FPLC (Fast Protein Liquid Chromatography) profile of the purification of the beta-xylosidase, correlated with the polyacrylamide electrophoresis gel containing sodium dodecyl sulfate (SDS PAGE, Bio-Rad, Mini-PROTEAN TGX Stain-Free Precast Gel 10%-456-8035), according to example 1. In this figure band 1 can be seen, corresponding to the enzyme purified by this chromatographic technique, which has been cut from the gel and analyzed by mass spectrometry. The protein identified by mass spectroscopy corresponds to the protein XP_006964075.1 in NCBI and with UniProtKB reference: Q92458_HYPJE already cited above. The same type of result is obtained with example 2.

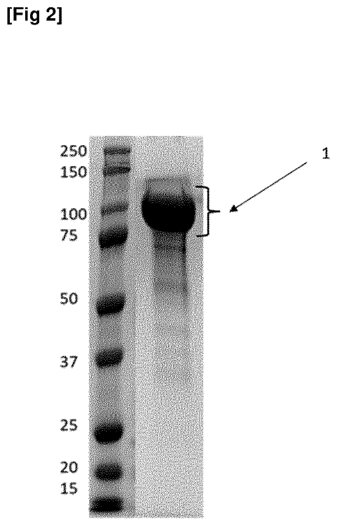

FIG. 2 is an image of the SDS-PAGE gel, for example 1, which makes it possible to separate the proteins according to their molecular masses after having denatured the proteins. In order to have an indication of the molecular mass, the left-hand band has markers for various molecular sizes: 15 kDa, 20 kDa, 25 kDa, 37 kDa, 50 kDa, 75 kDa, 100 kDa, 150 kDa and 250 kDa. On the right-hand band, the purified beta-xylosidase was loaded and then analyzed. The result of the electrophoresis of the beta-xylosidase with an SDS-PAGE gel shows that the molecular mass 1 corresponds to that predicted by the DNA sequence, i.e. of between 75 kDa and 100 kDa. In addition, it is found that the protein is pure. The same type of result is obtained with example 2.

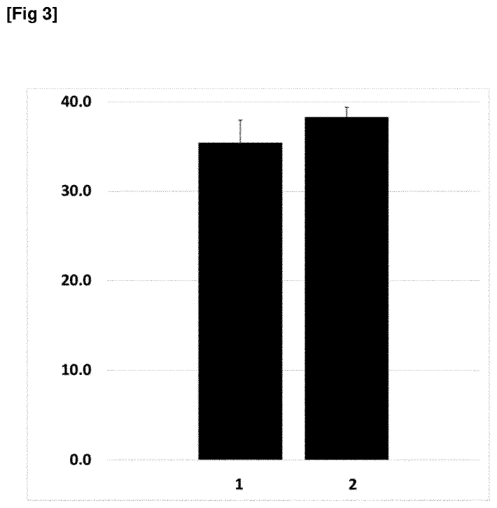

Following the various purifications, the activity was measured on different batches of purified enzymes, and the activity results are indicated in the bar chart of FIG. 3. The figure shows that the beta-xylosidase enzyme purified from examples 1 and 2 has a specific activity of approximately 35 to 38 μmol of p-nitrophenol·min⁻¹·mg⁻¹ of enzyme.

In conclusion, this technique for IMAC purification of the beta-xylosidase makes it possible to obtain this enzyme rapidly, simply and very efficiently, and without using a histidine tag. The affinity of the beta-xylosidase for the IMAC column is a discovery that could not have been expected. The beta-xylosidase separated according to the invention was identified by mass spectrometry, and the specific activity thereof, evaluated with para-nitrophenyl β-D-xylopyranoside (pNPX) as substrate, varies between at least 15 or 20 and 35 μmol of p-nitrophenol·min⁻¹·mg⁻¹ of enzyme or more.

This enzyme is of industrial interest for stimulating the degradation of xylan or oligomers of xylose with various degrees of polymerization (DP) such as xylobiose, xylotriose, or with a higher xylose DP.

It may be profitably exploited alone, or combined with other enzyme cocktails/mixtures depending on the needs and applications.

1. A process for separating beta-xylosidase enzymes (1) from a mixture of enzymes comprising beta-xylosidase enzymes and other enzymes, the process comprising:
separating from the rest of the enzyme mixture by immobilized metal ion affinity chromatography (MAC), and wherein the beta-x vlosidase enzymes to be separated are devoid of a histidine group.

2. The separation process as claimed in claim 1, wherein the other enzymes of said mixture comprise at least one enzyme chosen from cellulases and/or from hemicellulases.

3. The separation process as claimed in claim 1, wherein the other enzymes of said mixture comprise beta-glucosidases, endoglucanases, hemicellulases, and optionally cellobiohydrolases.

4. The separation process as claimed in claim 1, wherein the beta-xylosidases (1) constitute at least 1% by weight of all of the enzymes present in the mixture.

5. The separation process as claimed in claim 1, wherein the IMAC uses a solid immobile phase comprising a matrix of metal ions are fixed by chelating agents, and a liquid mobile phase referred to as eluent.

6. The separation process as claimed claim 1, wherein the matrix of the immobile phase is chosen from at least one of the following compounds: agarose gel, crosslinked dextran gel, and silica.

7. The separation process as claimed claim 5, wherein the chelating agents are chosen from iminodiacetic acid IDA, nitrolotriacetic acid NTA, and tris[carboxymethyl]-ethylenediamine TED.

8. The separation process as claimed in claim 5, wherein the metal ions are chosen from metal ions of transition metals.

9. The separation process as claimed in claim 1, wherein the enzyme mixture is obtained from production of enzymes by a microorganism.

10. The separation process as claimed in claim 1, further comprising a preliminary step of separating a culture medium comprising the enzyme mixture and a microorganism referred to as must that produced said mixture, said preliminary step being aimed at separating the must from said liquid enzyme mixture.

11. The separation process as claimed in claim 1, further comprising a step of treating the must, which may or may not have been separated from the rest of the culture medium, said treatment comprising cooling the must and then separating the must and a liquid containing an additional amount of enzyme mixture.

12. The separation process as claimed in claim 1, wherein the chromatography is carried out continuously in a chromatography column containing a solid immobile phase through which a liquid mobile phase referred to as eluent can be continuously passed.

13. The separation process as claimed in claim 1, wherein the separation by chromatography is carried out batchwise, by bringing an immobile chromatography phase into contact with the mixture comprising beta-xylosidase enzymes and other enzymes, in a liquid medium, to constitute a reaction medium in a container for a given duration, and then by eluting the solid part of said reaction medium in order to extract the beta-xylosidases therefrom.

14. The separation process as claimed in claim 1, wherein the separation comprises a step of mixing the immobile phase with the mixture of enzymes in solution, then an optional decantation step, then a step of isolating the solid phase from the reaction medium, then an optional washing step, then a step of eluting the isolated solid phase in order to extract the beta-xylosidases therefrom.

15. The separation process as claimed in claim 1, wherein the separation by chromatography fixes the beta-xylosidases (1) on the immobile phase at a pH of between 6.5 and 9, and in that the beta-xylosidases (1) are eluted by changing the nature, the composition or the concentration of the eluent.

16. The beta-xylosidase enzymes (1) obtained by the process as claimed in claim 1, wherein the enzymes have a specific activity of at least 10 μmol of p-nitrophenol·min⁻¹·mg⁻¹ of enzyme.

17. The beta-xylosidase enzymes (1) obtained by the process as claimed in claim 1, wherein the enzymes have a purity of greater than or equal to 90%.

18. A method of enriching an enzyme cocktail produced by a microorganism with beta-xylosidase enzymes, comprising:
separating from the enzyme cocktail by immobilized metal ion affinity chromatography (IMAC).

19. The separation process as claimed in claim 1, wherein the beta-xylosidases (1) constitute at between 2% and 15% by weight of all of the enzymes present in the mixture.

20. The separation process as claimed in claim 5, wherein the metal ions are chosen from: Cu(II), Ni(II), Zn(II), Co(II), Fe(III), Al(III), Ga(III), and Zr(IV).
